(19) United States
(12) Patent Application Publication
(10) Pub. No.: US 2025/0120573 A1
(43) Pub. Date: Apr. 17, 2025

(54) VALVE SEALS FOR ENDOSCOPES

(71) Applicant: Boston Scientific Scimed, Inc., Maple Grove, MN (US)

(72) Inventors: Brian Luis, Boylston, MA (US); Rosangel Aimee Ramos Espinoza, Boston, MA (US); Colby Harris, Norfolk, MA (US); Nathan Thomas Cummings, Worcester, MA (US)

(73) Assignee: Boston Scientific Scimed, Inc., Maple Grove, MN (US)

(21) Appl. No.: 18/910,590

(22) Filed: Oct. 9, 2024

Related U.S. Application Data
(60) Provisional application No. 63/589,863, filed on Oct. 12, 2023.

(51) Int. Cl. A61B 1/00

(52) U.S. Cl. CPC A61B 1/00137

(57) ABSTRACT

An endoscope (e.g., a duodenoscope) includes a bifluidic valve for controlling the flow of water and air through a flexible tubular probe to respectively irrigate or insufflate an internal cavity of a patient. Some examples of the bifluidic valve include a flexible valve seal that allows air to flow in one direction but not in reverse. Different examples of the valve seal include various features, such as a set of overlapping flexible flaps arranged in a spiral, a rubber-like disc with an array of cross-slit perforations that open in response to a positive pressure differential across the disc, a resiliently expandable cone with multiple concentric ridges, a valve spool encircled by an elastic tubular piece that can expand radially to open an air passageway in the valve spool, and an elastic tube that in reaction to being compressed or otherwise shortened bulges radially outward to block an annular air passageway.

### CROSS-REFERENCE TO RELATED APPLICATIONS

This application claims the benefit of U.S. Provisional Patent Application Ser. No. 63/589,863 filed on Oct. 12, 2023, the disclosure of which is incorporated herein by reference.

### TECHNICAL FIELD

Various embodiments of this disclosure relate generally to endoscopes (e.g., duodenoscopes, colonoscopes, bronchoscopes, etc.) and more specifically to valve seals for endoscopes.

### BACKGROUND

Endoscopes have revolutionized the field of medical diagnostics and interventions by enabling medical practitioners to directly visualize internal cavities of the human body without the need for invasive surgeries. Among the various types of endoscopes, duodenoscopes hold a prominent place due to their capability to explore the upper gastrointestinal tract, particularly the duodenum, pancreas, and bile ducts. Duodenoscopes facilitate not only visual examinations but also a range of therapeutic procedures, making them indispensable tools in modern medicine.

Duodenoscopes typically comprise a flexible tubular probe for inserting into the patient. A light source at a distal end of the probe provides illumination for viewing. Often a high-resolution camera is adjacent to the light source for capturing real-time images or videos of the internal cavities. The camera's capabilities ensure detailed and accurate observations.

A duodenoscope's probe usually includes a working channel, allowing for the insertion of various instruments for procedures like biopsies, tissue removal, or stent placement. To provide maneuverability and access to intricate anatomical structures, many duodenoscope probes have internal wires. The tension in the wires can be adjusted in opposing sets of two by manipulating knobs on a handle body of the duodenoscope. Adjusting the wire tension enables bending and steering of the probe.

Handle bodies typically include valves (e.g., one-way seal valves and bifluidic valves) for controlling fluid flow, such as air for insufflation and water for irrigation. Insufflation is a technique used during endoscopic procedures to improve visualization. It involves the introduction of air or carbon dioxide into the cavity being examined, which helps to expand the space, allowing for better visibility of the targeted area. In duodenoscopy, insufflation aids in the assessment of mucosal surfaces and the identification of abnormalities that might otherwise be obscured. This technique enhances the accuracy of diagnoses and assists in determining appropriate treatment strategies.

Irrigation typically involves the introduction of liquids, such as sterile water or saline. Irrigation serves several purposes during endoscopic procedures. It can help clear blood, debris, or mucus from the visual field, ensuring clear visibility. Irrigation can also aid in therapeutic interventions by flushing out areas of interest, allowing for better access and manipulation of tissues. This feature may enhance the safety and effectiveness of procedures like polyp removal or tissue sampling.

### SUMMARY

The present disclosure generally pertains to bifluidic valves for endoscopes, wherein the bifluidic valves are selectively configurable to an insufflating configuration and an irrigating configuration. In some examples, the bifluidic valve is further configurable to a vented configuration. Some examples of the bifluidic valve include a valve housing with an inner surface at least partially defining a housing interior. In some examples, the valve housing at least partially defines a gas inlet and a gas outlet 4. Some examples of the bifluidic valve include a valve spool that is elongate to define an axial direction. In some examples, the valve spool extends into the housing interior. In some examples, the valve spool and the inner surface define an annular gap therebetween. Some examples of the bifluidic valve include a valve seal extending across the annular gap in a radial direction between the valve spool and the inner surface. In some examples, the valve seal includes a plurality of flaps that are resiliently flexible between a deformed shape and a more relaxed shape. In some examples, the plurality of flaps are in the deformed shape when the bifluidic valve is in the insufflating configuration to connect the gas inlet in fluid communication with the gas outlet. In some examples, the plurality of flaps are in the more relaxed shape when the bifluidic valve is in the irrigating configuration to block fluid communication between the gas inlet and the gas outlet.

In some examples, the bifluidic valve may include a valve housing with an inner surface at least partially defining a housing interior. In some examples, the valve housing at least partially defining a gas inlet and a gas outlet 4. In some examples, bifluidic valve may include a valve spool that is elongate to define an axial direction. In some examples, the valve spool extends into the housing interior. In some examples, the valve spool and the inner surface define an annular gap therebetween. In some examples, the bifluidic valve may include a valve seal extending across the annular gap in a radial direction between the valve spool and the inner surface. In some examples, the valve seal is resiliently flexible between a deformed shape and a more relaxed shape. In some examples, the valve seal is in the deformed shape when the bifluidic valve is in the insufflating configuration to connect the gas inlet in fluid communication with the gas outlet. In some examples, the valve seal is in the more relaxed shape when the bifluidic valve is in the closed configuration to block fluid communication between the gas inlet and the gas outlet. In some examples, the bifluidic valve includes a plurality of concentric ridges on a surface of the valve seal, extending around the valve spool, and lying substantially perpendicular to the axial direction. In some examples, the plurality of concentric ridges are distributed over an axial length that is greater when the valve seal is in the deformed shape than in the more relaxed shape. In some examples, the valve seal has an outer radial periphery that is greater in the more relaxed shape than in the deformed shape.

In some examples, the bifluidic valve may include a valve housing with an inner surface at least partially defining a housing interior. In some examples, the valve housing at least partially defines a gas inlet and a gas outlet 4. Some examples of the bifluidic valve may include a valve spool that is elongate to define an axial direction. In some examples, the valve spool extends into the housing interior. In some examples, the valve spool defines a gas passageway between the gas inlet and the gas outlet 4. Some examples of the bifluidic valve may include a valve seal encircling the valve spool and being attached thereto. In some examples, the valve seal is resiliently expandable from a more relaxed shape to a deformed shape in response to a pressure differential between the gas inlet and the gas outlet exceeding a predetermined threshold. In some examples, when the bifluidic valve is in the insufflating configuration, the valve seal is in the deformed shape to open the gas passageway and thereby connect the gas inlet in fluid communication with the gas outlet. In some examples, when the bifluidic valve is in the irrigating configuration, the valve seal is in the more relaxed shape to obstruct the gas passageway and thereby block fluid communication between the gas inlet and the gas outlet.

In some examples, the bifluidic valve may include a valve housing with an inner surface at least partially defining a housing interior. In some examples, the valve housing at least partially defines a gas inlet and a gas outlet 4. In some examples, the bifluidic valve may include a sleeve supported by the valve housing within the housing interior. In some examples, the bifluidic valve may include a valve spool that is elongate to define an axial direction. In some examples, the valve spool extends through the sleeve and is movable in the axial direction relative to the sleeve selectively to a home position, a partially depressed position for the insufflating configuration, and a fully depressed position for the irrigating configuration. In some examples, the bifluidic valve may include a valve seal with a first end, a second end, and a radially expandable section therebetween. In some examples, the first end is substantially stationary relative to the sleeve. In some examples, the second end is relatively stationary relative to the valve spool. In some examples, the radially expandable section engages the inner surface of the valve housing when the valve spool is in the home position. In some examples, the radially expandable section is spaced apart from the inner surface when the valve spool is in the partially depressed position. In some examples, the radially expandable section is spaced apart from the inner surface when the valve spool is in the fully depressed position.

In some examples, the bifluidic valve may include a valve housing. In some examples, the bifluidic valve may include a valve spool extending into the valve housing and being movable relative to the valve housing selectively to a home position, a partially depressed position for the insufflating configuration, and a fully depressed position for the irrigating configuration. In some examples, the bifluidic valve may include a valve seal comprising a first end and a second end. In some examples, the valve seal has a seal length as measured from the first end to the second end. In some examples, the first end is substantially stationary relative to the valve housing. In some examples, the second end is substantially stationary relative to the valve spool. In some examples, the seal length is longer when the valve spool is in the fully depressed position. In some examples, the seal length is shorter when the valve spool is in the home position.

The preceding summary is provided to facilitate an understanding of some of the features of the present disclosure and is not intended to be a full description. A full appreciation of the disclosure can be gained by taking the entire specification, claims, drawings and abstract.

### BRIEF DESCRIPTION OF THE DRAWINGS

FIG. 1 is a perspective view of an example endoscope with a bifluidic valve incorporating various embodiments of the present disclosure.

FIG. 2 is a cross-sectional view of an example bifluidic valve in a home position and a vented configuration, wherein the bifluidic valve includes an example valve seal shown in its more relaxed shape, and the bifluidic valve and the valve seal include various embodiments of the present disclosure.

FIG. 3 is a cross-sectional view like FIG. 2 but showing the bifluidic valve in the home position and an insufflating configuration, while the valve seal is in a deformed shape.

FIG. 4 is a cross-sectional view like FIG. 2 but showing the bifluidic valve in the depressed position and an irrigating configuration, while the valve seal is in its more relaxed shape.

FIG. 5 is a perspective view of an example valve seal that can be used in the bifluidic valves of FIGS. 2-4, wherein the valve seal is in its more relaxed shape.

FIG. 6 is a top view of FIG. 5.

FIG. 7 is a right side view of FIG. 6.

FIG. 8 is an exploded perspective view and an assembled perspective of an example valve seal that can be used in the bifluidic valves of FIGS. 2-4.

FIG. 9 is a cross-sectional view of an example bifluidic valve in a home position and a vented configuration, wherein the bifluidic valve includes an example valve seal shown in its more relaxed shape, and the bifluidic valve and the valve seal include various embodiments of the present disclosure.

FIG. 10 is a cross-sectional view like FIG. 9 but showing the bifluidic valve in the home position and an insufflating configuration, while the valve seal is in a deformed shape.

FIG. 11 is a cross-sectional view like FIG. 2 but showing the bifluidic valve in the depressed position and an irrigating configuration, while the valve seal is in its more relaxed shape.

FIG. 12 is a right side view of the valve seal shown in FIG. 9, wherein the valve seal is in a more relaxed shape.

FIG. 13 is a left side view of the valve seal shown in FIG. 9.

FIG. 14 is a close-up view of FIG. 12.

FIG. 15 is a close-up view of FIG. 13.

FIG. 16 is a close-up view like FIG. 14 but showing the valve seal in a more open deformed shape.

FIG. 17 is a close-up view like FIG. 15 but showing the valve seal in a more open deformed shape.

FIG. 18 is a cross-sectional view taken along line 18-18 of FIG. 14.

FIG. 19 is a cross-sectional view taken along line 19-19 of FIG. 16.

FIG. 20 is a cross-sectional view like FIG. 18 but showing a second example of the valve seal.

FIG. 21 is a cross-sectional view like FIG. 19 but showing the second example of the valve seal.

FIG. 22 is a cross-sectional view of another example bifluidic valve in a home position and a vented configuration, wherein the bifluidic valve includes an example valve seal shown in its more relaxed shape, and the bifluidic valve and the valve seal include various embodiments of the present disclosure.

FIG. 23 is a cross-sectional view like FIG. 22 but showing the bifluidic valve in the home position and an insufflating configuration, while the valve seal is in a deformed shape.

FIG. 24 is a cross-sectional view like FIG. 22 but showing the bifluidic valve in the depressed position and an irrigating configuration, while the valve seal is in its more relaxed shape.

FIG. 25 is a radial cross-sectional view of the valve seal in its more relaxed shape.

FIG. 26 is a radial cross-sectional view of the valve seal in its deformed shape.

FIG. 27 is a radial cross-sectional view like FIG. 25 but showing a second example of the valve seal in its more relaxed shape.

FIG. 28 is a radial cross-sectional view of the second valve seal in its deformed shape.

FIG. 29 is a cross-sectional view of an example bifluidic valve in a home position and a vented configuration, wherein the bifluidic valve includes an example valve seal shown in its more relaxed shape, and the bifluidic valve and the valve seal include various embodiments of the present disclosure.

FIG. 30 is a cross-sectional view like FIG. 29 but showing the bifluidic valve in the home position and an insufflating configuration, while the valve seal is in a deformed shape.

FIG. 31 is a cross-sectional view like FIG. 29 but showing the bifluidic valve in the depressed position and an irrigating configuration, while the valve seal is in its more relaxed shape.

FIG. 32 is a cross-sectional view of another example bifluidic valve in a home position and a vented configuration, wherein the bifluidic valve includes an example valve seal shown in its more relaxed shape, and the bifluidic valve and the valve seal include various embodiments of the present disclosure.

FIG. 33 is a cross-sectional view like FIG. 32 but showing the bifluidic valve in the home position and an insufflating configuration, while the valve seal is in a deformed shape.

FIG. 34 is a cross-sectional view like FIG. 32 but showing the bifluidic valve in the depressed position and an irrigating configuration, while the valve seal is in its more relaxed shape.

FIG. 35 is a cross-sectional view of yet another example bifluidic valve in a home position and a vented configuration, wherein the bifluidic valve includes an example valve seal shown in its more relaxed shape, and the bifluidic valve and the valve seal include various embodiments of the present disclosure.

FIG. 36 is a cross-sectional view like FIG. 35 but showing the bifluidic valve in the home position and an insufflating configuration, while the valve seal is in a deformed shape.

FIG. 37 is a cross-sectional view like FIG. 35 but showing the bifluidic valve in the depressed position and an irrigating configuration, while the valve seal is in its more relaxed shape.

### DESCRIPTION

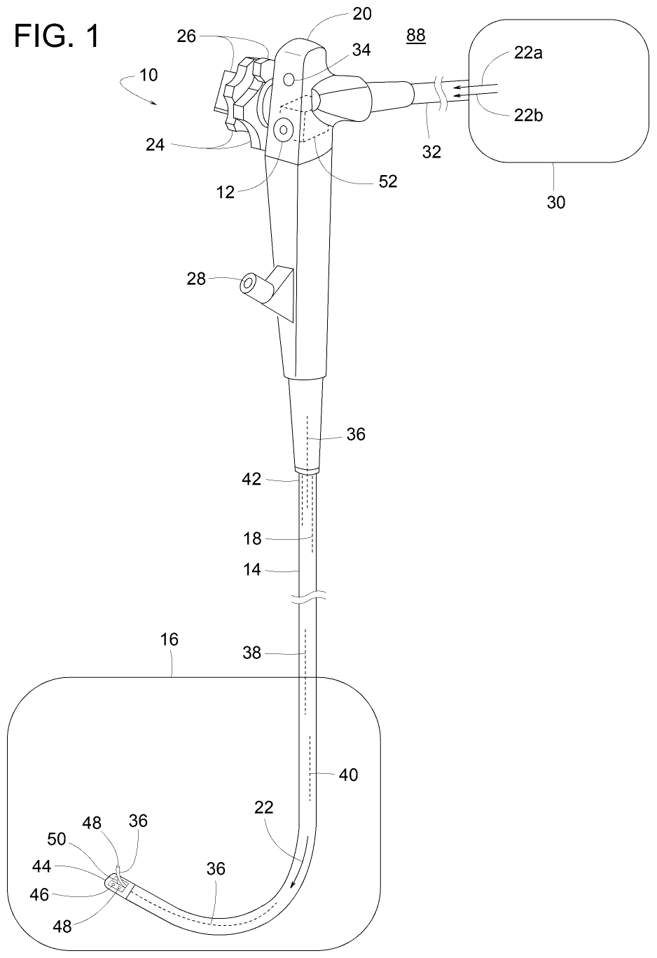

FIGS. 1-37 show various examples of a bifluidic valve 12 (e.g., a one-way seal valve) for an endoscope 10 and methods for using them. The term, “bifluidic” as it relates to a valve means that the valve can handle at least two streams of fluid, wherein one fluid is a gas, and the other fluid is a liquid. The valve can be a single component, an interconnected assembly of components, or a plurality of separate components. The term, “endoscope” represents any medical apparatus with a flexible tubular probe 14 for inserting into a patient 16 to visually explore the patient's internal tissues and cavities and to introduce water, air, or other fluids when desired. Some example endoscopes 10 have internal wires 18 with adjustable tension for bending and steering the flexible tubular probe 14. Some examples of the endoscope 10, shown in FIG. 1, include duodenoscopes, colonoscopes, ureteroscopes, bronchoscopes, laparoscopes, sheaths, and catheters.

The endoscope 10 is illustrated as an example, so many of the following listed components are optional. Some examples of the endoscope 10 include components such as a handle body 20, the flexible tubular probe 14 extending from the handle body 20, the bifluidic valve 12 (e.g., bifluidic valves 12a-f) for controlling the flow of fluid 22 (e.g., a liquid 22a and a gas 22b), steering knobs 24 to adjust the tension in the internal wires 18, locking levers 26 to lock the position of the steering knobs 24, a biopsy port 28 for sampling withdrawn tissue or fluid, a control unit 30, an umbilicus 32 connecting the control unit 30 to the handle body 20, and an image capture button 34.

The control unit 30 provides various functions. Some examples of such functions include supplying liquid 22a (e.g., water, saline, etc.), supplying gas 22b (e.g., air, carbon dioxide, etc.), sending and receiving electrical signals, processing electrical signals, providing a source of vacuum, etc. Some of the control unit's functions listed here are optional. The umbilicus 32 connects the control unit 30 in signal communication or fluid communication with the bifluidic valve 12, the flexible tubular probe 14, or other endoscope-related components.

In some examples, the flexible tubular probe 14 contains various components such as the internal wires 18 for steering, tubing 36 (one or more tubes) for conveying fluids 22, a fiber optic cable 38 for conveying images or light, and electrical wires 40 for conveying electrical power or signals. Some of these probe components are optional.

The flexible tubular probe 14 has a proximal end 42 and a distal end 44. The proximal end 42 connects to the handle body 20, and the distal end 44 extends away from the handle body 20. At the distal end 44, some examples of the flexible tubular probe 14 include a light 46 (or fiber optic cable leading thereto) for illuminating a patient's internal cavities, a camera 48 (or fiber optic cable leading thereto), a tip 48 of the tubing 36, and an elevator 50 for tilting the tip 48 of the tubing 36. The elevator 50 is also known as a swing stand, a pivot stand, and a raising bed. The tip 48 of the tubing 36 is open to pass fluid 22 for insufflating, irrigating, or biopsy sampling.

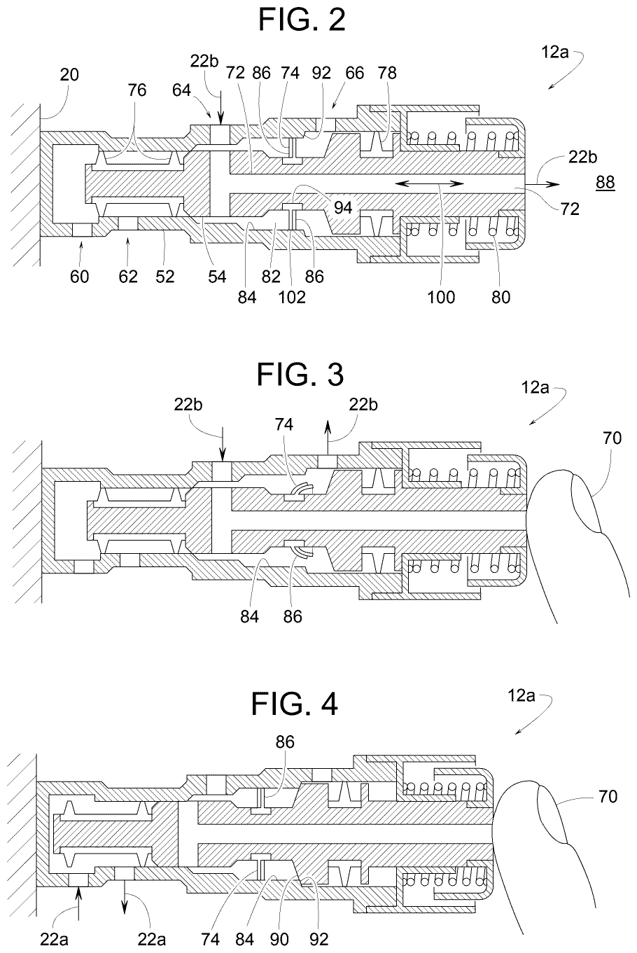

Insufflating and irrigating can be controlled in different ways by various examples of the bifluidic valve 12 (e.g., bifluidic valves 12a-f). In the example shown in FIGS. 2-8, the bifluidic valve 12a is configurable selectively to a vented configuration (FIG. 2), an insufflating configuration (FIG. 3) and an irrigating configuration (FIG. 4). The vented configuration can prevent or minimize the release of gas 22b out through the flexible tubular probe 14. The insufflating configuration can be used for delivering gas 22b down through the flexible tubular probe 14 and out through the distal end 44 to expand a body cavity for better observation. The irrigating configuration can be used for flushing certain areas with liquid 22a during examination. To select a desired configuration, some examples of the bifluidic valve 12a include a valve housing 52 in which a valve spool 54 can be moved lengthwise in an axial direction 100 between a home position (FIGS. 2 and 3) and a depressed position (FIG. 4).

In some examples of bifluidic valve 12a, the valve spool 54 has a vent 72 that a user 70 (e.g., a medical practitioner) can manually cover or uncover. When the vent 72 is uncovered while the valve spool 54 is in the home position, as shown in FIG. 2, the bifluidic valve 12a is in the vented configuration. When the vent 72 is covered while the valve spool 54 is in the home position, as shown in FIG. 3, the bifluidic valve 12a is in the insufflating configuration. When the vent 72 is covered while the valve spool 54 is in the depressed position, as shown in FIG. 4, the bifluidic valve 12a is in the irrigating configuration.

Some examples of bifluidic valve 12a comprise the valve housing 52, the valve spool 54, a valve seal 74, a first set of seals 76, a second seal 78, and a spring 80. The spring 80 urges the valve spool 54 to its home position. In some examples, the valve seal 74 encircles the valve spool 54 and is attached thereto. In some examples, the valve seal 74 extends radially across an annular gap 82 between the OD (outside diameter) of the valve spool 54 and the ID (inside diameter) of the valve housing's inner surface 84.

In some examples, the first set of seals 76 is carried by the valve spool 54 and is positioned within the valve housing 52 to control the flow of liquid 22a through the bifluidic valve 12a. In some examples, the second seal 78 on valve spool 54 is to help prevent any fluid 22 from leaking out of the bifluidic valve 12a between the OD of the valve spool 54 and the ID of the valve housing 52.

The valve housing 52 is supported by the handle body 20, as shown in FIGS. 2-4. The means for support is shown schematically, as the valve housing 52 can be connected in any desired way and at any location and orientation relative to the handle body 20. In some examples, the valve housing 52 is a seamless integral extension of the handle body 20.

The valve housing 52 is shown as a seamless unitary piece (i.e., a single part rather than an assembly of parts); however, other examples of valve housing 52 comprise a plurality of parts. In some examples, the plurality parts are interconnected as an assembly. In some examples, the plurality of parts are separated and spaced apart from each other. In some examples, the valve spool 54 is made of metal (e.g., stainless steel, brass, bronze, titanium, aluminum, etc.) while the valve housing 52 is made of a polymer such as, for example, nylon, PEEK (polyetheretherketone), polycarbonate, ABS (acrylonitrile-butadiene-styrene), HDPE (high-density polyethylene), UHMW (ultra-high molecular-weight polyethylene), POM (polyoxymethylene, polyacetal, Delrin, Celcon, etc.), POM-C (polyoxymethylene copolymer), and POM-H (polyoxymethylene homopolymer). Such a combination of metal and polymeric materials can provide an accurate fit and low friction between the valve spool 54 and the valve housing 52.

Some examples of the valve housing 52 at least partially define or otherwise provide a liquid inlet 60, a liquid outlet 62, a gas inlet 64, and a gas outlet 66. The terms, “inlet” and “outlet” refer to areas through which a fluid can pass. Some examples of inlets and outlets include valve ports, valve orifices, tubes connected to a valve, fluid-conveying chambers within a valve, etc.

The liquid inlet 60 of the valve housing 52 receives liquid 22a from the control unit 30 via umbilicus 32. The tubing 36 within the flexible tubular probe 14 connects the liquid outlet 62 to the probe's distal end 44. Likewise, the gas inlet 64 of the valve housing 52 receives gas 22b from the control unit 30 via umbilicus 32. Tubing 36 within the flexible tubular probe 14 connects the gas outlet 66 to the probe's distal end 44.

The axial position of the valve spool 54 within the valve housing 52 determines whether the liquid 22a can flow from the liquid inlet 60 to the liquid outlet 62. When the valve spool 54 is in the home position (FIGS. 2 and 3), the first set of seals 76 carried by the valve spool 54 is positioned within the valve housing 52 to block liquid 22a from flowing through the bifluidic valve 12a. When the valve spool 54 is in the depressed position (FIG. 4), the location of the first set of seals 76 connects the liquid inlet 60 in fluid communication with the liquid outlet 62, so the liquid 22a can flow through the bifluidic valve 12a and be released out through the distal end 44 of the flexible tubular probe 14.

In some examples, the axial position of the valve spool 54 within the valve housing 52 and whether the vent 72 is covered determines whether the valve seal 74 allows the gas 22b to flow from the gas inlet 64 to the gas outlet 66. In some examples, the gas 22b at the gas inlet 64 is at about 5-9 psig. In some examples, the valve seal 74, specifically its shape, determines whether the gas 22b can flow by it. In some examples, the valve seal 74 resiliently expands from a more relaxed shape (FIGS. 2 and 4-7) to a deformed shape (FIG. 3) in response to a positive pressure differential between the gas inlet 64 and the gas outlet 66 exceeding a predetermined threshold (e.g., a positive 0.1 psi). So, a positive pressure differential (pressure at gas inlet 64 being greater than at gas outlet 66) basically pushes the valve seal 74 open. A negative pressure differential (pressure at gas inlet 64 being less than at gas outlet 66), however, forces the valve seal 74 closed, preventing undesirable backflow.

In some examples, the valve seal 74 includes a plurality of flaps 86 that are resiliently flexible between the deformed shape (FIG. 3) and the more relaxed shape (FIGS. 2 and 4-7). In some examples, the plurality of flaps 86 are in the more relaxed shape (FIGS. 2 and 5-7) when the bifluidic valve 12a is in the vented configuration (FIG. 2) to prevent or minimize the release of gas 22a out through the flexible tubular probe 14. In the vented configuration, the pressure at the gas inlet 64 is relatively low (near atmospheric pressure), as the vent 72 connects the gas inlet 64 in fluid communication with atmosphere 88 (i.e., the ambient air around the endoscope 10). Thus, the pressure differential between the gas inlet 64 and the gas outlet 66 is insufficient to open the valve seal 74.

In some examples, the plurality of flaps 86 are in the deformed shape when the bifluidic valve 12a is in the insufflating configuration (FIG. 3), connecting the gas inlet 64 in fluid communication with the gas outlet 66. In the insufflating configuration, the valve spool 54 is in the home position and the vent 72 is covered. With the vent 72 closed, the pressure at the gas inlet 64 can rise above atmospheric pressure and push the valve seal 74 open. The gas 22a is then free to flow in series through the gas inlet 60, past the valve seal 74, through the gas outlet 66, through the flexible tubular probe 14, and out through the probe's distal end 44.

In some examples, the plurality of flaps 86 are in the more relaxed shape (FIGS. 4-7) when the bifluidic valve 12a is in the irrigating configuration (FIG. 4). In the irrigating configuration, a shoulder 90 on the valve spool 54 seals against a valve seat 92 of the valve housing 52. The valve spool's shoulder 90 sealing against the valve seat 92 blocks fluid communication between the gas inlet 64 and the gas outlet 66. This causes the pressure differential across the valve seal 74 to equalize, so the valve seal 74 settles to its more relaxed shape, as shown in FIG. 4.

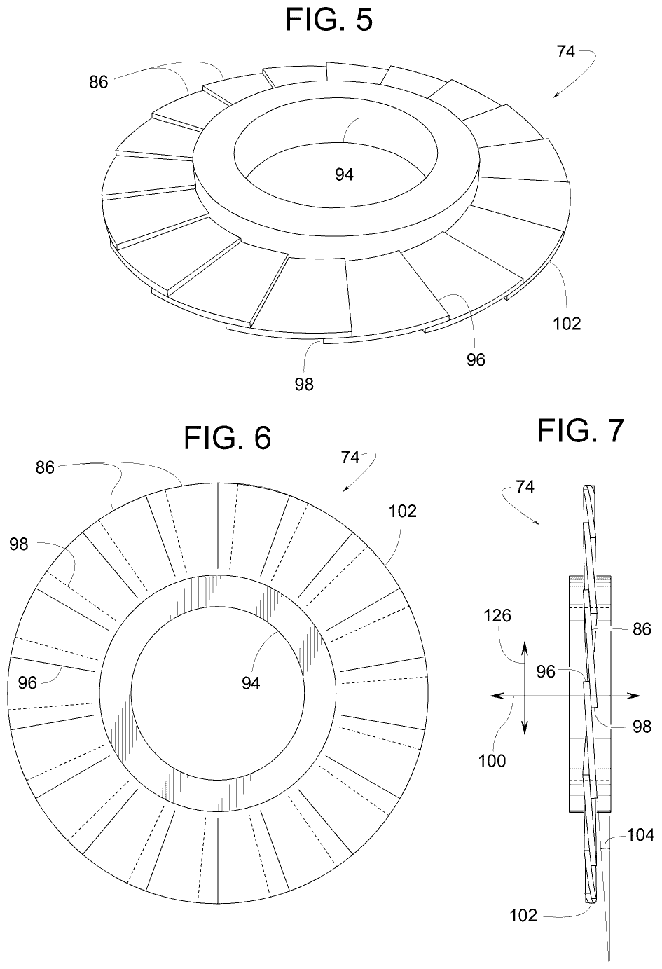

In some examples, the valve seal's plurality of flaps 86 are shaped and arranged as shown in FIGS. 5-8. The illustrated example shows the valve seal 74 with eighteen flaps 86. Other examples of the valve seal 74 have more than eighteen. Other examples have less than eighteen.

In the example shown in FIGS. 2-8, an inner periphery 94 of the valve seal 74 is attached to the valve spool 54, while the plurality of flaps 86 are distributed in a circular pattern around the valve spool 54. Each flap 86 includes an inlet edge 96 and an outlet edge 98. Each inlet edge 96 faces or is otherwise exposed to the gas inlet 64. Each outlet edge 98 faces or is otherwise exposed to the gas outlet 66. So, the inlet and outlet edges 96 and 98 are on opposite faces of the valve seal 74. The plurality of flaps 86 are in an overlapping arrangement such that the inlet edge 96 of each flap 86 overlies the outlet edge 98 of an adjacent flap 86. This places the plurality of flaps 86 in overlapping sliding contact with each other at the inlet and outlet edges 96 and 98. The sliding contact of the plurality of flaps 86 provides the valve seal 74 with freedom to flex in an axial direction 100 without undue hoop strain along an outer periphery 102 of the valve seal 74.

In the deformed shape, the seal's outer periphery 102 is spaced apart from the valve housing's inner surface 84, as shown in FIG. 3. In the more relaxed shape (FIGS. 5-7), the outer periphery 102 engages the inner surface 84, as shown in FIGS. 2 and 4.

In some examples, the valve seal 74 is plastic injection molded followed by a second operation of slicing the valve seal 74 between the overlapping edges of adjacent flaps 86. In some examples, an angle of slicing 104 is greater near the inner periphery 94 than at the outer periphery 102 for the same reason a screw's helical angle is greater near the thread's root diameter than at the thread's outer diameter.

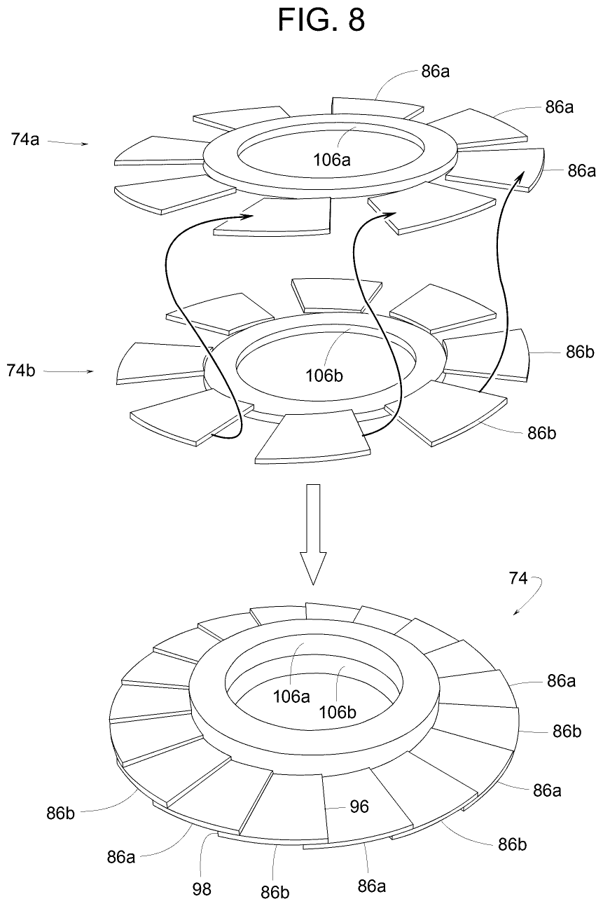

Although the valve seal 74 might seem challenging to make, it can be done. In some examples, as shown in FIG. 8, the valve seal 74 is plastic injection molded in two parts, a first half 74a and a second half 74b. The first half 74a comprises a first half hub 106a with an integral plurality of flaps 86a. Likewise, the second half 74b comprises a second half hub 106b with an integral plurality of flaps 86b. After injection molding, the two halves 74a and 74b are brought together back-to-back and rotated a few degrees so the two halves 74a and 74b interlock with their respective flaps 86a and 86b overlapping. In some examples, the two halves 74a and 74b are injection molded in separate mold cavities and assembled afterwards. In some examples, the first half 74a is overmolded onto the second half 74b in the same mold cavity. In some examples, the first and second halves 74a and 74b are identical. In some examples, the first and second halves 74a and 74b are made of different materials to facilitate overmolding one onto the other.

In some examples, the flaps 86 are made of a rubber-like material, such a silicone or TPE (thermoplastic elastomer). In some examples, the material thickness of the flaps 86 is sufficiently thin to be made of stiffer material. In some examples, the valve seal 74 is overmolded onto the valve spool 54 in a manner like some examples of the first set of seals 76 and the second seal 78. In some examples, the flaps 86 are substantially coplanar and lie edge-to-edge, rather than in an overlapping arrangement.

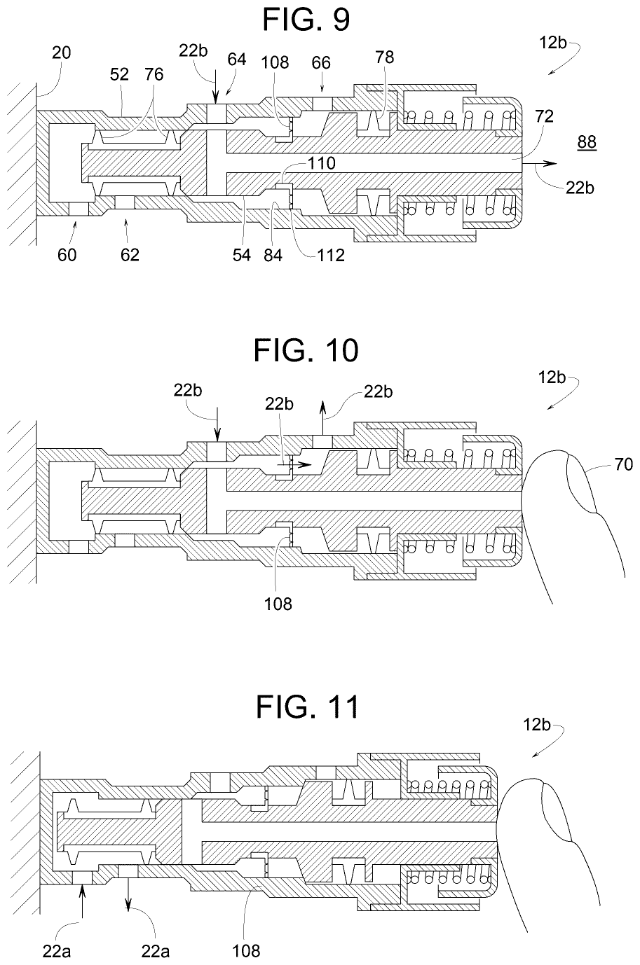

FIGS. 9-11 show the example bifluidic valve 12b, which is similar in many ways to the bifluidic valve 12a. Bifluidic valve 12b includes the same valve housing 52 and valve spool 54 as bifluidic valve 12a. Like the bifluidic valve 12a, the valve spool 54 in bifluidic valve 12b is movable to a home position (FIGS. 9 and 10) and a depressed position (FIG. 11). The vent 72 in the bifluidic valve 12b can be manually covered (FIGS. 10 and 11) or uncovered (FIG. 9). Depending on the position of the valve spool 54 and whether the vent 72 is covered or uncovered, the bifluidic valve 12b is configurable selectively to a vented configuration (FIG. 9), an insufflating configuration (FIG. 10), or an irrigating configuration (FIG. 14).

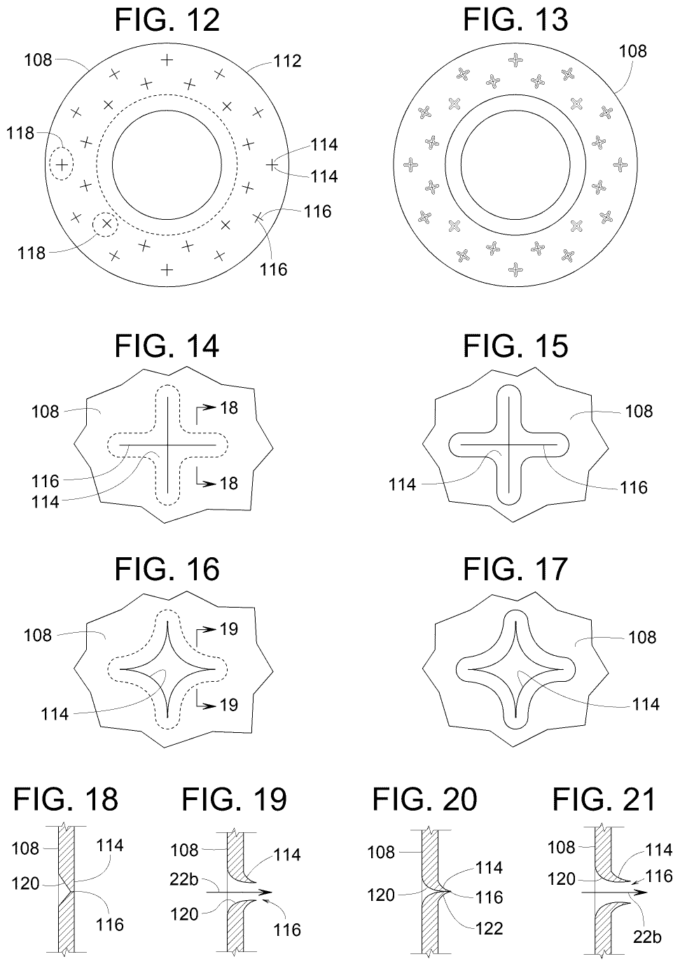

Nonetheless, some examples of the bifluidic valve 12b have a different valve seal 108, as shown in FIGS. 12-21. In the illustrated examples, the valve seal 108 has an inner periphery 110 attached to the valve spool 54 and an outer periphery 112 that stays in sliding sealing contact with the ID of a valve housing 52.

The valve seal 108, in some examples, includes a plurality of flaps 114 defining a plurality of slits 116, as shown in FIGS. 12-21. Each slit 116 is more open for the gas 22a to flow through when the plurality of flaps 114 are in the deformed shape (FIGS. 10, 16, 17, 19 and 21) than when the plurality of the flaps 114 are in the more relaxed shape (FIGS. 9, 11-15, 18 and 20).

In some examples, the plurality of flaps 114 are separated in a plurality of groups 118 such that each group 118 defines a cluster of intersecting slits 116. Each cluster of intersecting slits 116 is spaced apart from the inner periphery 110 and the outer periphery 112 to minimize leakage in those areas. The illustrated examples have four flaps 114 per each group 118. Other examples have less than four flaps 114 per group 118, e.g., three flaps per group 118. Still other examples have more than four flaps 114.

The plurality of flaps 114 and slits 116 function under a one-way flow principle like that of a conventional duckbill valve, wherein the slits 116 open in response to a sufficient positive pressure differential being applied across the valve seal 108. In some examples, the flaps 114 and slits 116 include a tapered inlet 120 to promote the opening of the slits 116 in response to the pressure differential. In some examples, the flaps 114 and slits 116 include a protruding outlet 122 that further promotes the opening of the slits 116.

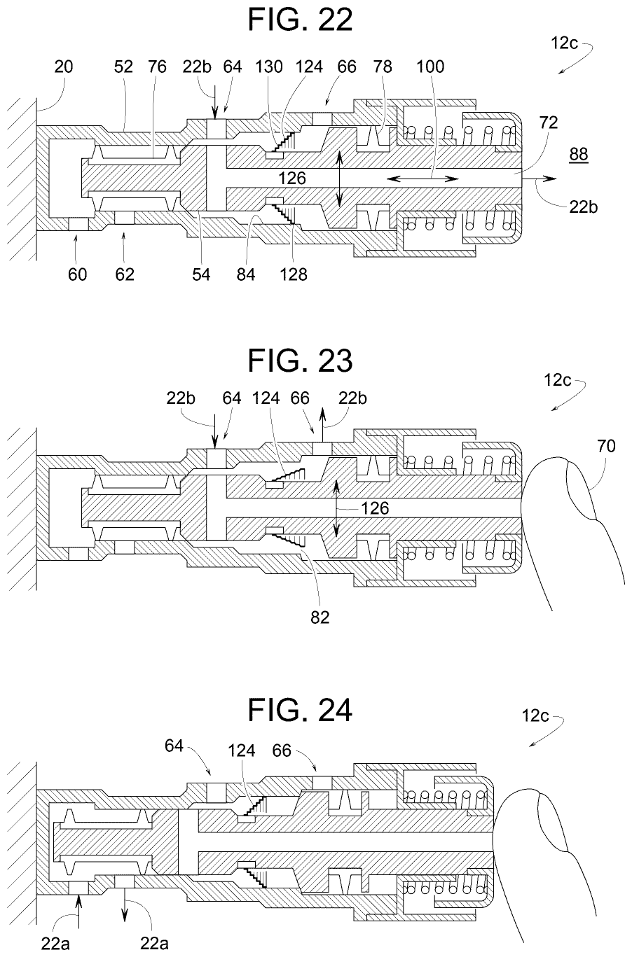

FIGS. 22-24 show the example bifluidic valve 12c, which is similar in many ways to the bifluidic valves 12a. Bifluidic valve 12c includes the same valve housing 52 and valve spool 54 as bifluidic valve 12a. Like the bifluidic valve 12a, the valve spool 54 in bifluidic valve 12c is movable to a home position (FIGS. 22 and 23) and a depressed position (FIG. 24). The vent 72 in the bifluidic valve 12c can be manually covered (FIGS. 23 and 24) or uncovered (FIG. 22). Depending on the position of the valve spool 54 and whether the vent 72 is covered or uncovered, the bifluidic valve 12c is configurable selectively to a vented configuration (FIG. 22), an insufflating configuration (FIG. 23), or an irrigating configuration (FIG. 24). In the various configurations, the control of fluid flow through the bifluidic valve 12c is as described with reference to bifluidic valve 12a.

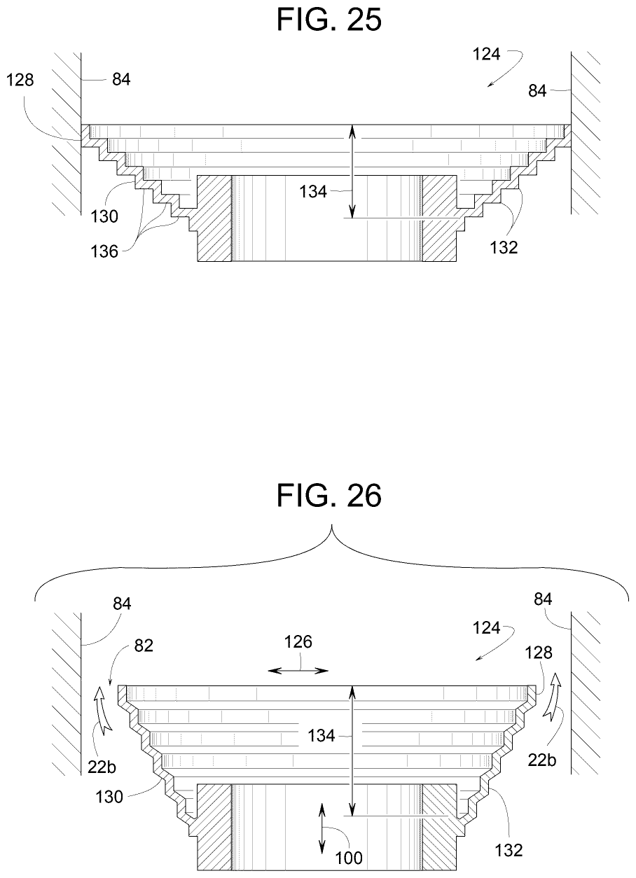

Some examples of the bifluidic valve 12c have a different valve seal 124, as shown in FIGS. 25 and 26. The valve seal 124 extends across the annular gap 82 in a radial direction 126 between the valve spool 54 and the housing's inner surface 84. The valve seal 124 is resiliently flexible between a deformed shape (FIGS. 23 and 26) and a more relaxed shape (FIGS. 22, 24 and 25).

A sufficient pressure differential between the gas inlet 64 and the gas outlet 66 is what pushes the valve seal 124 to its deformed shape. In the deformed shape, an outer periphery 128 of the valve seal 124 is radially spaced apart from the valve housing's inner surface 84, so the gas 22b can flow past the valve seal 124 between the valve's outer periphery 128 and the valve housing's inner surface 84. The valve seal 124 is in the deformed shape when the bifluidic valve 12c is in the insufflating configuration (FIG. 23), wherein the gas inlet 64 is in fluid communication with the gas outlet 66.

In the more relaxed shape, when the pressure differential across the valve seal 124 is below the predetermined threshold (e.g., below 0.1 psi), the outer periphery 128 of the valve seal 124 sealingly engages the valve housing's inner surface 84 to block fluid communication in either direction between the gas inlet 64 and the gas outlet 66. The valve seal 124 is in the more relaxed shape (FIGS. 22 and 24) when the bifluidic valve 12c is in the closed or vented configuration.

To increase flexibility and reduce hoop strain at the seal's outer periphery 128, some examples of the valve seal 124 include a surface 130 with a plurality of concentric ridges 132. The ridges 132 extend around the valve spool 54 and lie substantially perpendicular to the axial direction 100. The term, “substantially perpendicular,” as it relates to the ridges 132, means that each concentric ridge 132 lies along an imaginary plane that is within 10 degrees of being perpendicular to the axial direction 100. The plurality of ridges 132 are distributed over an axial length 134 that is greater when the valve seal 124 is in the deformed shape (FIGS. 23 and 26) than in the more relaxed shape (FIGS. 22, 24 and 25). To effectively seal radially against the valve housing's inner surface 84, the valve seal's outer radial periphery 128 is greater in the more relaxed shape than in the deformed shape.

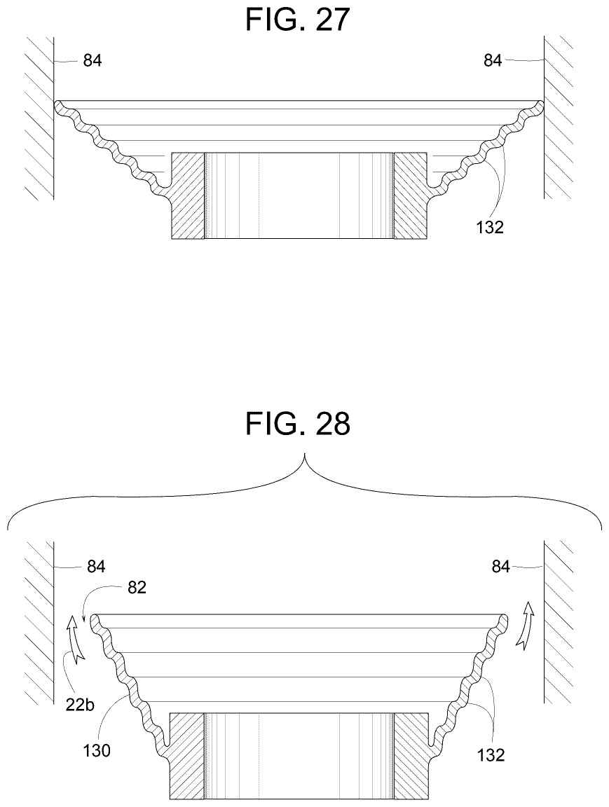

In some examples, as shown in FIGS. 25 and 26, the ridges 132 are created by virtue of the valve seal's surface 130 having a plurality of incremental steps 136. In some examples, as shown in FIGS. 27 and 28, the ridges 132 are the result of the valve seal's surface 130 being wavy.

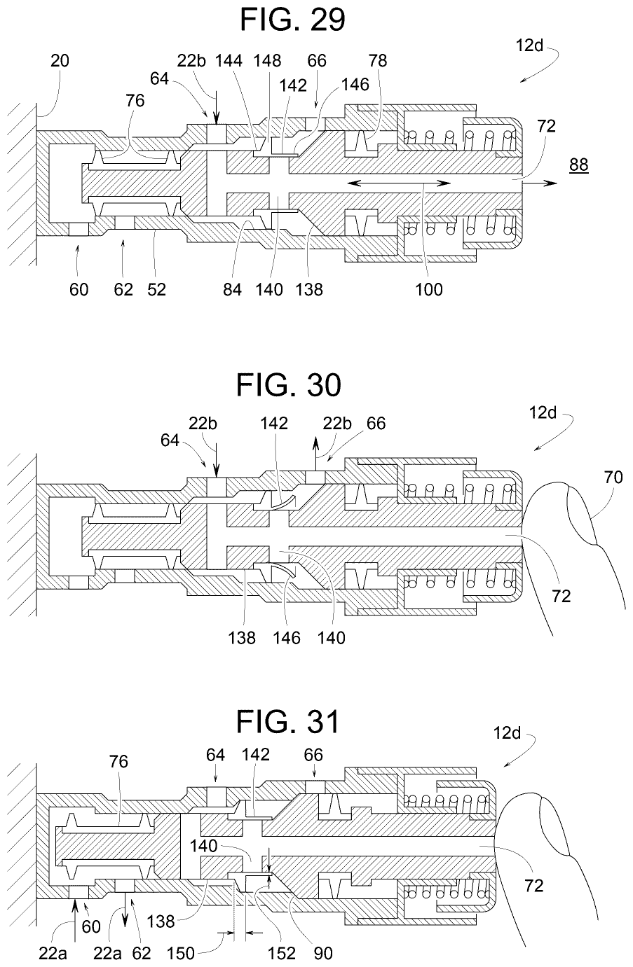

FIGS. 29-31 show an example of bifluidic valve 12d. In some examples, bifluidic valve 12d includes a valve spool 138 that is moveable within the valve housing 52 between a home position (FIGS. 29 and 30) and a depressed position (FIG. 31). The valve spool 138 has a gas passageway 140 for connecting the gas inlet 64 in fluid communication with the gas outlet 66 when the bifluidic valve 12d is in an insufflating configuration (FIG. 30).

In some examples, the valve spool 138 has the vent 72 connected in fluid communication with the gas passageway 140. In some examples, the vent 72 can be manually covered (FIGS. 30 and 31) to isolate the gas passageway 140 from atmosphere 88, or the vent 72 can be uncovered (FIG. 29) to open the gas passageway 140 to atmosphere 88.

The bifluidic valve 12d also includes a valve seal 142. The valve seal 142 encircles the valve spool 138 and is attached to it. The valve seal 142 is resiliently expandable from a more relaxed shape (FIGS. 29 and 31) to a deformed shape (FIG. 30) in response to a pressure differential between the gas inlet 64 and the gas outlet 66 exceeding a predetermined threshold (e.g., 0.1 psi).

Depending on the position of the valve spool 138 and whether the vent 72 is covered or uncovered, the bifluidic valve 12d is configurable selectively to a vented configuration (FIG. 29), an insufflating configuration (FIG. 30), and an irrigating configuration (FIG. 31). In the various configurations, the control of fluid flow through the bifluidic valve 12d is like that which was described with reference to bifluidic valve 12a.

The valve seal 142 of the bifluidic valve 12d, however, comprises a fixed end 144, a movable end 146, and a flange 148. In some examples, the valve seal 142, including the fixed end 144, the movable end 146, and the flange 148; is a seamless unitary piece to minimize leakage and manufacturing costs. The fixed end 144 is substantially stationary relative to the valve spool 138. The term, “substantially stationary,” as it relates to two parts means that there no relative sliding, rotation, or translation of the two parts other than perhaps some deflection, stretching, compressing, twisting, or other strain. The movable end 146 is flexible and movable relative to the valve spool 138, such that the movable end 146 is closer to the valve spool 138 when the valve seal 142 is in the more relaxed shape (FIGS. 29 and 31) than when the valve seal 142 is in the deformed shape (FIG. 30).

The flange 148 is at the fixed end 144 of the valve seal 142 and extends radially from the valve spool 138, so the flange 148, the fixed end 144 and the valve spool 138 move as a unit. Thus, the flange 148, the fixed end 144 and the valve spool 138 are movable in the axial direction 100 between the home position (FIGS. 29 and 30) and the depressed position (FIG. 31), relative to the valve housing 52.

As the valve spool 138 moves, the flange 148 slides in sealing contact along the inner surface 84 of the valve housing 52. The sealing contact encourages any gas 22b flowing from the gas inlet 64 to the gas outlet 66 to flow through the gas passageway 140, rather than bypassing the gas passageway 140 by flowing past the flange 148. In some examples, the flange 148 has an axial thickness 150 that is greater than a radial wall thickness 152 of the valve seal's movable end 146. The relative thicknesses ensure that the flange 148 can resist a significant pressure differential while enabling the movable end 146 to flex as needed.

When the bifluidic valve 12d is in the vented configuration, as shown in FIG. 29, the vent 72 connects the gas passageway 140 to atmosphere 88. This leaves insufficient pressure in the gas passageway 140 to deform the valve seal 142, thus the valve seal 142 remains in its more relaxed shape. In the more relaxed shape, the valve seal 142 blocks gas 22b from flowing in either direction between the gas inlet 64 and gas outlet 66, so virtually no gas 22b gets delivered to the flexible tubular probe 14. Also, the flange 148 prevents the gas 22b from bypassing the valve seal 142.

When the bifluidic valve 12d is in the insufflating configuration, as shown in FIG. 30, the manually obstructed vent 72 allows the pressure in the gas passageway 140 to equalize with the pressure in the gas inlet 64. This creates a sufficient pressure differential between the gas passageway 140 and the gas outlet 66 to force open the valve seal 142, which allows the gas 22b to flow in series from the gas inlet 64, through the gas passageway 140, through the deflected valve seal 142, through the gas outlet 66, and out through the distal end 44 of the flexible tubular probe 14.

When the bifluidic valve 12d is in the irrigating configuration, as shown in FIG. 31, the first set of seals 76 are positioned to connect the liquid inlet 60 in fluid communication with the liquid outlet 62, allowing the liquid 22a to flow in series from the liquid inlet 60, through the liquid outlet 62, and out through the distal end 44 of the flexible tubular probe 14. In the irrigating configuration, the valve spool's shoulder 90 sealing against the valve seat 92 blocks fluid communication between the gas inlet 64 and the gas outlet 66. This causes the pressure differential across the valve seal 142 to equalize, so the valve seal 142 returns to its more relaxed shape.

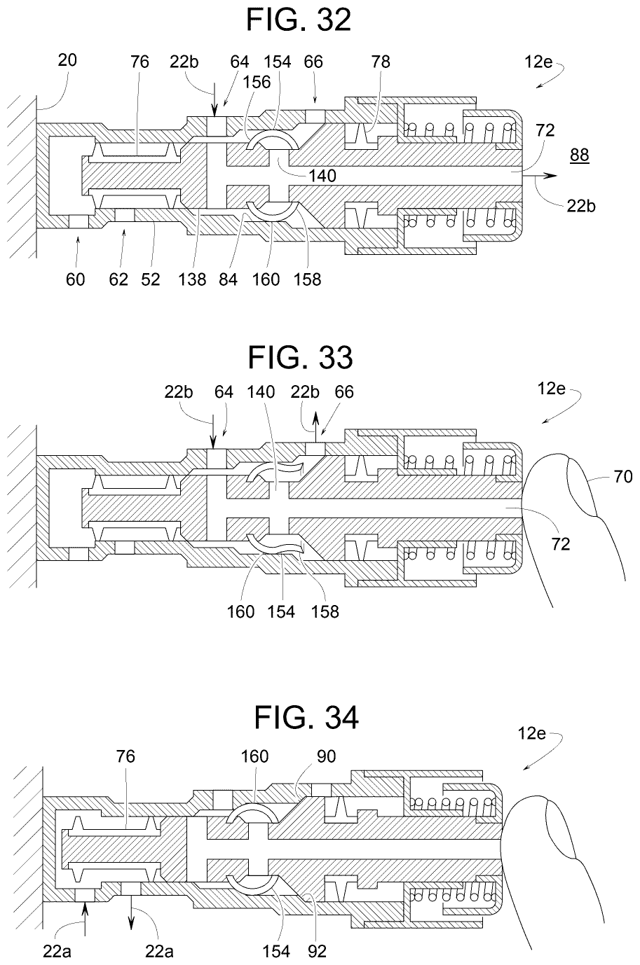

FIGS. 32-34 show an example of bifluidic valve 12e. In some examples, bifluidic valve 12e includes the valve spool 138 that is moveable within the valve housing 52 between a home position (FIGS. 32 and 33) and a depressed position (FIG. 34). The valve spool 138 has the gas passageway 140 for connecting the gas inlet 64 in fluid communication with the gas outlet 66 when the bifluidic valve 12e is in an insufflating configuration (FIG. 32).

In some examples, the valve spool 138 has the vent 72 connected in fluid communication with the gas passageway 140. In some examples, the vent 72 can be manually covered (FIGS. 33 and 34) to isolate the gas passageway 140 from atmosphere 88, or the vent 72 can be uncovered (FIG. 32) to open the gas passageway 140 to atmosphere 88.

The bifluidic valve 12e also includes a valve seal 154. The valve seal 154 encircles the valve spool 138 and is attached to it. The valve seal 154 is resiliently expandable from a more relaxed shape (FIGS. 32 and 34) to a deformed shape (FIG. 33) in response to a pressure differential between the gas inlet 64 and the gas outlet 66 exceeding a predetermined threshold (e.g., 0.1 psi).

Depending on the position of the valve spool 138 and whether the vent 72 is covered or uncovered, the bifluidic valve 12e is configurable selectively to a vented configuration (FIG. 32), an insufflating configuration (FIG. 33), and an irrigating configuration (FIG. 34). In the various configurations, the control of fluid flow through the bifluidic valve 12e is like that which was described with reference to bifluidic valves 12a and 12d.

The valve seal 154 of the bifluidic valve 12e, however, comprises a fixed end 156, a movable end 158, and a bulging section 160 axially interposed therebetween. The fixed end 156 is substantially stationary relative to the valve spool 138. The movable end 158 is flexible and movable relative to the valve spool 138, such that the movable end 158 is closer to the valve spool 138 when the valve seal 154 is in the more relaxed shape (FIGS. 32 and 34) than when the valve seal 154 is in the deformed shape (FIG. 33). The bulging section 160 sealingly engages the valve housing's inner surface 84. In some examples, the valve seal 154, including the fixed end 156, the movable end 158, and the bulging section 160; is a seamless unitary piece to minimize leakage and manufacturing costs.

As the valve spool 138 moves, the bulging section 160 slides in sealing contact along the inner surface 84 of the valve housing 52. The sealing contact encourages any gas 22b flowing from the gas inlet 64 to the gas outlet 66 to flow through the gas passageway 140, rather than bypassing the gas passageway 140 around the bulging section 160.

When the bifluidic valve 12e is in the vented configuration, as shown in FIG. 32, the vent 72 connects the gas passageway 140 to atmosphere 88. This leaves insufficient pressure in the gas passageway 140 to deform the valve seal 154, thus the valve seal 154 remains in its more relaxed shape. In the more relaxed shape, the valve seal 154 blocks gas 22b from flowing in either direction between the gas inlet 64 and gas outlet 66, so virtually no gas 22b gets delivered to the flexible tubular probe 14. Also, the bulging section 160 prevents the gas 22b from bypassing the valve seal 154.

When the bifluidic valve 12e is in the insufflating configuration, as shown in FIG. 33, the manually obstructed vent 72 allows the pressure in the gas passageway 140 to equalize with the pressure in the gas inlet 64. This creates a sufficient pressure differential between the gas passageway 140 and the gas outlet 66 to force open the valve seal 154, which allows the gas 22b to flow in series from the gas inlet 64, through the gas passageway 140, through the deflected valve seal 154, through the gas outlet 66, and out through the distal end 44 of the flexible tubular probe 14.

When the bifluidic valve 12e is in the irrigating configuration, as shown in FIG. 34, the first set of seals 76 are positioned to connect the liquid inlet 60 in fluid communication with the liquid outlet 62, allowing the liquid 22a to flow in series from the liquid inlet 60, through the liquid outlet 62, and out through the distal end 44 of the flexible tubular probe 14. In the irrigating configuration, the valve spool's shoulder 90 sealing against the valve seat 92 blocks fluid communication between the gas inlet 64 and the gas outlet 66. This causes the pressure differential across the valve seal 154 to equalize, so the valve seal 154 returns to its more relaxed shape.

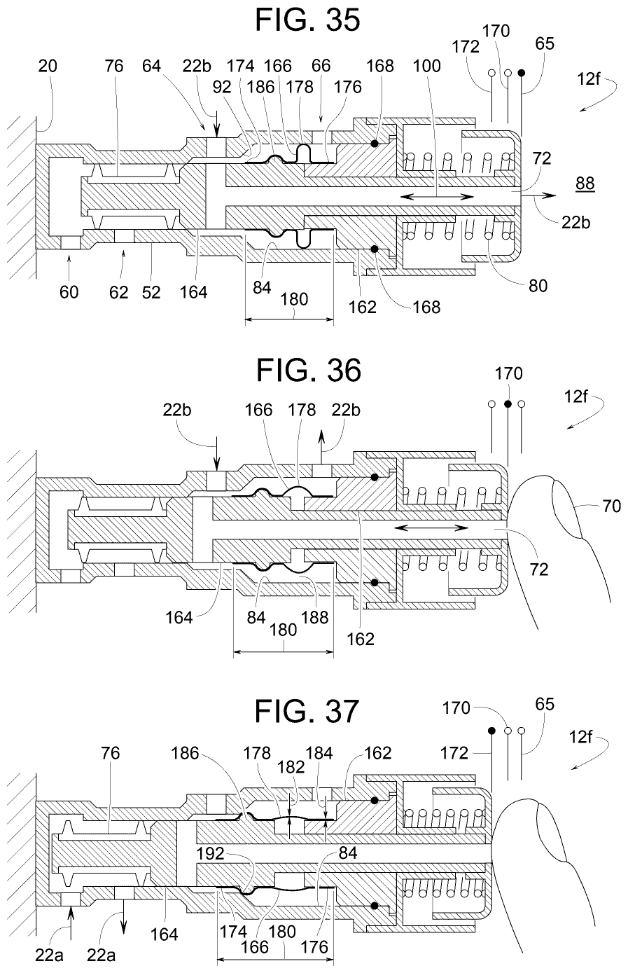

FIGS. 34-36 show an example of bifluidic valve 12f that can be configured selectively to a vented configuration (FIG. 35), an insufflating configuration (FIG. 36), and an irrigating configuration (FIG. 37). In some examples, bifluidic valve 12f comprises the valve housing 52, a sleeve 162 fixed within the valve housing 52, a valve spool 164 extending through the sleeve 162, and a valve seal 166. In some examples, a combination of the valve housing 52 and the sleeve 162 is a seamless unitary piece.

In the illustrated example, points 168 schematically represents any interface between the valve housing 52 and the sleeve 162. Some examples of interfaces represented by the points 168 include O-rings, sealants, adhesives, an interference fit between the valve housing 52 and the sleeve 162, a threaded connection between the valve housing 52 and the sleeve 162, a chemically fused joint, an ultrasonically welded joint, or an integral seamless connection between the valve housing 52 and the sleeve 162.

In examples where the points 168 represent an integral seamless connection, the combination of the valve housing 52 and the sleeve 162 is a seamless unitary piece. In other examples, the valve housing 52 and the sleeve 162 are discrete components to facilitate assembly of the bifluidic valve 12f.

In some examples, the valve spool 164 is elongate in the axial direction 100. Some examples of the valve spool 164 include the vent 72 that the user 70 can manually cover (FIGS. 36 and 37) or uncover (FIG. 35). In some examples, the valve spool 164 is movable in the axial direction 100 relative to the sleeve 162. In some examples, the valve spool 164 is movable selectively to a home position 65 (FIG. 34), a partially depressed position 170 for the insufflating configuration (FIG. 36), and a fully depressed position 172 for the irrigating configuration (FIG. 37).

In some examples, the valve seal 166 is an elastomeric tube comprises a first end 174, a second end 176, and a radially expandable section 178 therebetween. The valve seal 166 has a seal length 180 as measured from the first end 174 to the second end 176. The first end 174 is attached to the valve spool 164, so the first end 174 is substantially stationary relative to the valve spool 164. The second end 176 is attached to the sleeve 162, so the second end 176 is relatively stationary relative to the sleeve 162 and the valve housing 52.

The valve spool 164 moving axially relative to the valve housing 52 changes the length 180 of the valve seal 166. The seal's length 180 is shorter when the valve spool 164 is in the home position (FIG. 35). The seal's length 180 is longer when the valve spool 164 is in the fully depressed position (FIG. 37). The seal's length 180 is at an intermediate length when the valve spool 164 is in the partially depressed position (FIG. 36).

The radially expandable section 178 radially expands, as the seal's length 180 gets shorter. Conversely, the radially expandable section 178 radially retracts, as the seal's length 180 gets longer. In some examples, the radially expandable section 178 sealingly engages the valve housing's inner surface 84 when the valve spool 164 is in the home position 65, as shown in FIG. 35. The radially expandable section 178 is spaced apart from the inner surface 84 when the valve spool 164 is in either the partially depressed position 170 (FIG. 36) or the fully depressed position 172 (FIG. 37).

In some examples, the radially expandable section 178 has a material wall thickness 182 that is thinner than the material wall thickness 184 of the first and second ends 174 and 176. This helps ensure that the valve seal 166 expands in the desired area along the valve seal 166. In some examples, the valve seal's first end 174 extends over a shoulder 186 on the valve spool 164, so when the bifluidic valve 12f is in the irrigating configuration (FIG. 37), the relatively soft valve seal 166 provides a more compliant sealing surface between the shoulder 186 and the valve seat 92 on the valve housing 52.

In some examples, the radially expandable section 178 is in a relatively relaxed state when the valve seal 166 is in the shape shown in FIG. 35. In such examples, the valve spool 164 moving from the home position 65 to the fully depressed position 172 stretches the valve seal 166 in the axial direction 100. In some examples where the valve spool 164 stretches the valve seal 166 as the valve spool 164 is depressed, the axial resilience of the valve seal 166 provides a restorative force that can be used for urging the valve spool 164 back to its home position 65. A surprising and unexpected result is achieved in examples where the restorative force is so strong that the spring 80 is no longer needed and can be eliminated. In other words, the valve seal 166 can also serve as a return spring.

In other examples, the radially expandable section 178 is in a relatively relaxed state when the valve seal 166 is in the shape shown in FIG. 37. In such examples, the valve spool 164 moving from the fully depressed position 172 to the home position 65 compresses the valve seal 166 in the axial direction 100.

In still other examples, the radially expandable section 178 is in a relatively relaxed state when the valve seal 166 is in the shape shown in FIG. 36. In such examples, the valve spool 164 moving from the partially depressed position 170 to the home position 65 compresses the valve seal 166 in the axial direction 100 and moving from the partially depressed position 170 to the fully depressed position 172 stretches the valve seal 166 in the axial direction 100.

When the bifluidic valve 12f is in the vented configuration with the valve spool 164 is in the home position 65, as shown in FIG. 35, the seal's radially expandable section 178 sealingly engages the valve housing's inner surface 84, thereby blocking the gas 22b from flowing in either direction between the gas inlet 64 and the gas outlet 66. Additionally, the vent 72 connects the gas inlet 64 to atmosphere 76, which minimizes the pressure in the gas inlet 64, thus reducing the likelihood of the gas 22b in the gas inlet 64 being able to force itself past the valve seal 166. Thus, virtually no gas 22b gets delivered to the flexible tubular probe 14.

When the bifluidic valve 12f is in the insufflating configuration with the valve spool 164 in the partially depressed position 170, as shown in FIG. 36, the manually obstructed vent 72 prevents the gas 22b in the gas inlet 64 from escaping to atmosphere 88. Instead, when the valve spool 164 is partially depressed, the valve seal's radially expandable section 178 retracts from the valve housing's inner surface 84. This creates an annular gap 188 that allows gas 22b to flow from the gas inlet 64 to the gas outlet 66 and out through the distal end 44 of the flexible tubular probe 14.

When the bifluidic valve 12f is in the irrigating configuration with the valve spool 164 in the fully depressed position 172, as shown in FIG. 37, the first set of seals 76 are positioned to connect the liquid inlet 60 in fluid communication with the liquid outlet 62, allowing the liquid 22a to flow in series from the liquid inlet 60, through the liquid outlet 62, and out through the distal end 44 of the flexible tubular probe 14. In the irrigating configuration, the valve spool's shoulder 186 sealing against the valve seat 92 blocks fluid communication between the gas inlet 64 and the gas outlet 66. This restricts gas 22b from flowing to the flexible tubular probe 14.

It should be noted that the valve seal 166 provides a non-sliding hermetic seal between the moving valve spool 164 and the valve housing 52. A surprising and unexpected result of such a design is that the sliding second seal 78 used in bifluidic valves 12a-e is no longer needed and can be omitted in bifluidic valve 12f, while providing superior sealing at lower cost.

It should also be noted that any of the seals shown in FIGS. 2-37 can be made of any suitable sealing material. Some example sealing materials include silicone, TPE (thermoplastic elastomer), TPU (thermoplastic polyurethane), TPR (thermoplastic rubber), TPC (thermoplastic co-polyesters), PTFE (polytetrafluorethlylene), VersaFlex, neoprene, latex, nitrile, and any other flexible or rubber-like material known to those of ordinary skill in the art. In some examples, the sealing material has a durometer of about 50.

Some examples of the bifluidic valves 12a-f can be defined as described in the following examples 1-29.

Example-1 A bifluidic valve for use in an endoscope, the bifluidic valve being configurable selectively to an insufflating configuration and an irrigating configuration, the bifluidic valve comprising: a valve housing with an inner surface at least partially defining a housing interior, the valve housing at least partially defining a gas inlet and a gas outlet 4; a valve spool being elongate to define an axial direction, the valve spool extending into the housing interior, the valve spool and the inner surface defining an annular gap therebetween; and a valve seal extending across the annular gap in a radial direction between the valve spool and the inner surface, the valve seal comprising a plurality of flaps that are resiliently flexible between a deformed shape and a more relaxed shape, the plurality of flaps being in the deformed shape when the bifluidic valve is in the insufflating configuration to connect the gas inlet in fluid communication with the gas outlet, the plurality of flaps being in the more relaxed shape when the bifluidic valve is in the irrigating configuration to block fluid communication between the gas inlet and the gas outlet.

Example-2 The bifluidic valve of Example-1, wherein the valve seal includes an inner periphery and an outer periphery, inner periphery is attached to the valve spool, the outer periphery is spaced apart from the inner surface when the plurality of flaps are in the deformed shape, and the outer periphery engages the inner surface when the flaps are in the more relaxed shape.

Example-3 The bifluidic valve of Example-1, wherein the plurality of flaps are distributed in a circular pattern around the valve spool, each flap of the plurality of flaps includes an inlet edge and an outlet edge, the inlet edge of each flap overlies the outlet edge of an adjacent flap of the plurality of flaps, the inlet edge of each flap is exposed to the gas inlet, the outlet edge of each flap is exposed to the gas outlet, the plurality of flaps are in overlapping sliding contact with each other at the inlet and outlet edges to facilitate the plurality of flaps flexing between the deformed shape and the more relaxed shape.

Example-4 The bifluidic valve of Example-1, wherein the valve housing at least partially defines a liquid inlet and a liquid outlet, the valve spool is movable in the axial direction relative to the valve housing between a home position and a depressed position, the valve spool in the depressed position configures the bifluidic valve to the irrigating configuration to establish fluid communication between the liquid inlet and the liquid outlet, the valve spool in the home position obstructs fluid communication between the liquid inlet and the liquid outlet, and the valve seal moves in the axial direction with the valve spool as the valve spool moves between the home position and the depressed position.

Example-5 The bifluidic valve of Example-4, wherein the valve spool at least partially defines a vent that can be selectively covered and uncovered manually, the vent when uncovered connects the gas inlet in fluid communication with atmosphere, the vent when covered blocks fluid communication between the gas inlet and the atmosphere, and the bifluidic valve is configured in a vented configuration when the vent is uncovered while the valve spool is in the home position.

Example-6 The bifluidic valve of Example-1, wherein the valve seal includes an inner periphery attached to the valve spool and an outer periphery engaging the inner surface of the valve housing, the plurality of flaps define a plurality of slits that are spaced apart from the inner periphery and the outer periphery, each slit of the plurality of slits being more open when the plurality of flaps are in the deformed shape than when the plurality of the flaps are in the more relaxed shape.

Example-7 The bifluidic valve of Example-1, wherein the valve seal includes an inner periphery attached to the valve spool and an outer periphery engaging the inner surface of the valve housing, the plurality of flaps are separated in a plurality of groups such that each group of the plurality of groups defines a cluster of intersecting slits that are spaced apart from the inner periphery and the outer periphery, each cluster of intersecting slits being more open when the plurality of flaps are in the deformed shape than when the plurality of the flaps are in the more relaxed shape.

Example-8 A bifluidic valve for use in an endoscope, the bifluidic valve being configurable selectively to an insufflating configuration and an irrigating configuration, the bifluidic valve comprising: a valve housing with an inner surface at least partially defining a housing interior, the valve housing at least partially defining a gas inlet and a gas outlet 4; a valve spool being elongate to define an axial direction, the valve spool extending into the housing interior, the valve spool and the inner surface defining an annular gap therebetween; a valve seal extending across the annular gap in a radial direction between the valve spool and the inner surface, the valve seal being resiliently flexible between a deformed shape and a more relaxed shape, the valve seal being in the deformed shape when the bifluidic valve is in the insufflating configuration to connect the gas inlet in fluid communication with the gas outlet, the valve seal being in the more relaxed shape when the bifluidic valve is in the closed configuration to block fluid communication between the gas inlet and the gas outlet; and a plurality of concentric ridges on a surface of the valve seal, extending around the valve spool, and lying substantially perpendicular to the axial direction; the plurality of concentric ridges being distributed over an axial length that is greater when the valve seal is in the deformed shape than in the more relaxed shape, the valve seal having an outer radial periphery that is greater in the more relaxed shape than in the deformed shape.

Example-9 The bifluidic valve of Example-8, wherein the surface of the valve seal comprises a plurality of incremental steps that create the plurality of concentric ridges.

Example-10 The bifluidic valve of Example-8, wherein the surface of the valve seal is wavy to create the plurality of concentric ridges.

Example-11 The bifluidic valve of Example-8, wherein the valve housing at least partially defines a liquid inlet and a liquid outlet, the valve spool is movable in the axial direction relative to the valve housing between home position and depressed position, the valve spool in the depressed position establishes fluid communication between the liquid inlet and the liquid outlet to configure the bifluidic valve in the irrigating configuration, the valve spool in the home position obstructs fluid communication between the liquid inlet and the liquid outlet, and the valve seal moves in the axial direction with the valve spool as the valve spool moves between the home position and the depressed position.

Example-12 The bifluidic valve of Example-11, wherein the valve spool at least partially defines a vent that can be selectively covered and uncovered manually, the vent when uncovered connects the gas inlet in fluid communication with atmosphere, the vent when covered blocks fluid communication between the gas inlet and the atmosphere, and the bifluidic valve is configured in a vented configuration when the vent is uncovered while the valve spool is in the home position.

Example-13 A bifluidic valve for use in an endoscope, the bifluidic valve being configurable selectively to an insufflating configuration and an irrigating configuration, the bifluidic valve comprising: a valve housing with an inner surface at least partially defining a housing interior, the valve housing at least partially defining a gas inlet and a gas outlet 4; a valve spool being elongate to define an axial direction, the valve spool extending into the housing interior, the valve spool defining a gas passageway between the gas inlet and the gas outlet 4; a valve seal encircling the valve spool and being attached thereto, the valve seal being resiliently expandable from a more relaxed shape to a deformed shape in response to a pressure differential between the gas inlet and the gas outlet exceeding a predetermined threshold; when the bifluidic valve is in the insufflating configuration, the valve seal is in the deformed shape to open the gas passageway and thereby connect the gas inlet in fluid communication with the gas outlet; and when the bifluidic valve is in the irrigating configuration, the valve seal is in the more relaxed shape to obstruct the gas passageway and thereby block fluid communication between the gas inlet and the gas outlet.

Example-14 The bifluidic valve of Example-13, wherein the valve seal includes a fixed end that is substantially stationary relative to the valve spool, the valve seal includes a movable end that is closer to the valve spool when the valve seal is in the more relaxed shape than when the valve seal is in the deformed shape.

Example-15 The bifluidic valve of Example-13, wherein the valve seal further comprises a flange extending radially from the valve spool, the valve spool is movable in the axial direction between a home position and a depressed position relative to the valve housing, and the flange is in sliding contact with the inner surface of the valve housing when the valve spool moves between the home position and the depressed position.

Example-16 The bifluidic valve of Example-13, wherein the valve seal includes a fixed end that is substantially stationary relative to the valve spool, a movable end that is closer to the valve spool when the valve seal is in the more relaxed shape than when the valve seal is in the deformed shape, and a flange extending radially from the valve spool and contacting the inner surface of the valve housing, and further wherein the valve seal with the fixed end, the movable end and the flange is a seamless unitary piece.

Example-17 The bifluidic valve of Example-16, wherein the flange has an axial thickness that is greater than a radial wall thickness of the valve seal at the movable end.

Example-18 The bifluidic valve of Example-13, wherein the valve seal includes a fixed end, a movable end, and a bulging section axially interposed therebetween, the fixed end is substantially stationary relative to the valve spool, the movable end is closer to the valve spool when the valve seal is in the more relaxed shape than when the valve seal is in the deformed shape, and the bulging section engages the inner surface of the valve housing.

Example-19 The bifluidic valve of Example-18, wherein the valve seal with the fixed end, the movable end and the bulging section is a seamless unitary piece.

Example-20 The bifluidic valve of Example-13, wherein the valve housing at least partially defines a liquid inlet and a liquid outlet, the valve spool is movable in the axial direction between a home position and a depressed position relative to the valve housing, the valve spool in the depressed position establishes fluid communication between the liquid inlet and the liquid outlet, and the valve spool in the home position obstructs fluid communication between the liquid inlet and the liquid outlet.

Example-21 The bifluidic valve of Example-20, wherein the valve spool at least partially defines a vent that can be selectively covered and uncovered manually, the vent when uncovered connects the gas inlet in fluid communication with atmosphere, the vent when covered blocks fluid communication between the gas inlet and the atmosphere, and the bifluidic valve is configured in a vented configuration when the vent is uncovered while the valve spool is in the home position.

Example-22 A bifluidic valve for use in an endoscope, the bifluidic valve being configurable selectively to an insufflating configuration and an irrigating configuration, the bifluidic valve comprising: a valve housing with an inner surface at least partially defining a housing interior, the valve housing at least partially defining a gas inlet and a gas outlet 4; a sleeve supported by the valve housing within the housing interior; a valve spool being elongate to define an axial direction, the valve spool extending through the sleeve and being movable in the axial direction relative to the sleeve selectively to a home position, a partially depressed position for the insufflating configuration, and a fully depressed position for the irrigating configuration; and a valve seal comprising a first end, a second end, and a radially expandable section therebetween; the first end being substantially stationary relative to the sleeve, the second end being relatively stationary relative to the valve spool, the radially expandable section engaging the inner surface of the valve housing when the valve spool is in the home position, the radially expandable section being spaced apart from the inner surface when the valve spool is in the partially depressed position, the radially expandable section being spaced apart from the inner surface when the valve spool is in the fully depressed position.

Example-23 The bifluidic valve of Example-22, wherein the radially expandable section expands radially outward against the inner surface of the valve housing in response to valve spool moving from the partially depressed position to the home position.

Example-24 The bifluidic valve of Example-22, wherein a combination of the sleeve and the valve housing is a seamless unitary piece.

Example-25 The bifluidic valve of Example-22, wherein each of the first end, the second end, and the radially expandable section have a material wall thickness, and the material wall thickness of each of the first end and the second end is greater than that of the radially expandable section.

Example-26 The bifluidic valve of Example-22, wherein the valve spool defines a vent that can be selectively covered and uncovered manually, the vent connecting the gas inlet in fluid communication with atmosphere when the vent is uncovered, and the gas inlet being blocked from fluid communication with atmosphere when the vent is covered.

Example-27 The bifluidic valve of Example-22, wherein the valve housing at least partially defines a water inlet and a water outlet, the water inlet being connected in fluid communication with the water outlet when the valve spool is in the fully depressed position, the valve spool blocking fluid communication between the water inlet and the water outlet when the valve spool is in the partially depressed position.

Example-28 A bifluidic valve for use in an endoscope, the bifluidic valve being configurable selectively to an insufflating configuration to convey a gas through the bifluidic valve, and an irrigating configuration to convey a liquid through the bifluidic valve, the bifluidic valve comprising: a valve housing; a valve spool extending into the valve housing and being movable relative to the valve housing selectively to a home position, a partially depressed position for the insufflating configuration, and a fully depressed position for the irrigating configuration; and a valve seal comprising a first end and a second end, the valve seal having a seal length as measured from the first end to the second end, the first end being substantially stationary relative to the valve housing, the second end being substantially stationary relative to the valve spool, the seal length being longer when the valve spool is in the fully depressed position, and the seal length being shorter when the valve spool is in the home position.

Example-29 The valve seal of Example-28, wherein the valve seal includes a radially expandable section between the first end and the second end, the radially expandable section engaging the valve housing when the valve spool is in the home position, the radially expandable section being spaced apart from the valve housing when the valve spool is in the partially depressed position, the radially expandable section being spaced apart from the valve housing when the valve spool is in the fully depressed position.

The disclosure should not be considered limited to the examples described above. Various modifications, equivalent processes, as well as numerous structures to which the disclosure can be applicable will be readily apparent to those of ordinary skill in the art upon review of the instant specification.

1. A bifluidic valve for use in an endoscope, the bifluidic valve being configurable selectively to an insufflating configuration and an irrigating configuration, the bifluidic valve comprising:
a valve housing with an inner surface at least partially defining a housing interior, the valve housing at least partially defining a gas inlet and a gas outlet;
a valve spool being elongate to define an axial direction, the valve spool extending into the housing interior, the valve spool and the inner surface defining an annular gap therebetween; and
a valve seal extending across the annular gap in a radial direction between the valve spool and the inner surface, the valve seal comprising a plurality of flaps that are resiliently flexible between a deformed shape and a more relaxed shape, the plurality of flaps being in the deformed shape when the bifluidic valve is in the insufflating configuration to connect the gas inlet in fluid communication with the gas outlet, the plurality of flaps being in the more relaxed shape when the bifluidic valve is in the irrigating configuration to block fluid communication between the gas inlet and the gas outlet.

2. The bifluidic valve of claim 1, wherein the valve seal includes an inner periphery and an outer periphery, inner periphery is attached to the valve spool, the outer periphery is spaced apart from the inner surface when the plurality of flaps are in the deformed shape, and the outer periphery engages the inner surface when the flaps are in the more relaxed shape.

3. The bifluidic valve of claim 1, wherein the plurality of flaps are distributed in a circular pattern around the valve spool, each flap of the plurality of flaps includes an inlet edge and an outlet edge, the inlet edge of each flap overlies the outlet edge of an adjacent flap of the plurality of flaps, the inlet edge of each flap is exposed to the gas inlet, the outlet edge of each flap is exposed to the gas outlet, the plurality of flaps are in overlapping sliding contact with each other at the inlet and outlet edges to facilitate the plurality of flaps flexing between the deformed shape and the more relaxed shape.

4. The bifluidic valve of claim 1, wherein the valve housing at least partially defines a liquid inlet and a liquid outlet, the valve spool is movable in the axial direction relative to the valve housing between a home position and a depressed position, the valve spool in the depressed position configures the bifluidic valve to the irrigating configuration to establish fluid communication between the liquid inlet and the liquid outlet, the valve spool in the home position obstructs fluid communication between the liquid inlet and the liquid outlet, and the valve seal moves in the axial direction with the valve spool as the valve spool moves between the home position and the depressed position.

5. The bifluidic valve of claim 4, wherein the valve spool at least partially defines a vent that can be selectively covered and uncovered manually, the vent when uncovered connects the gas inlet in fluid communication with atmosphere, the vent when covered blocks fluid communication between the gas inlet and the atmosphere, and the bifluidic valve is configured in a vented configuration when the vent is uncovered while the valve spool is in the home position.

6. The bifluidic valve of claim 1, wherein the valve seal includes an inner periphery attached to the valve spool and an outer periphery engaging the inner surface of the valve housing, the plurality of flaps define a plurality of slits that are spaced apart from the inner periphery and the outer periphery, each slit of the plurality of slits being more open when the plurality of flaps are in the deformed shape than when the plurality of the flaps are in the more relaxed shape.

7. The bifluidic valve of claim 1, wherein the valve seal includes an inner periphery attached to the valve spool and an outer periphery engaging the inner surface of the valve housing, the plurality of flaps are separated in a plurality of groups such that each group of the plurality of groups defines a cluster of intersecting slits that are spaced apart from the inner periphery and the outer periphery, each cluster of intersecting slits being more open when the plurality of flaps are in the deformed shape than when the plurality of the flaps are in the more relaxed shape.

8. A bifluidic valve for use in an endoscope, the bifluidic valve being configurable selectively to an insufflating configuration and an irrigating configuration, the bifluidic valve comprising:
a valve housing with an inner surface at least partially defining a housing interior, the valve housing at least partially defining a gas inlet and a gas outlet 4;
a valve spool being elongate to define an axial direction, the valve spool extending into the housing interior, the valve spool and the inner surface defining an annular gap therebetween;
a valve seal extending across the annular gap in a radial direction between the valve spool and the inner surface, the valve seal being resiliently flexible between a deformed shape and a more relaxed shape, the valve seal being in the deformed shape when the bifluidic valve is in the insufflating configuration to connect the gas inlet in fluid communication with the gas outlet, the valve seal being in the more relaxed shape when the bifluidic valve is in the closed configuration to block fluid communication between the gas inlet and the gas outlet; and
a plurality of concentric ridges on a surface of the valve seal, extending around the valve spool, and lying substantially perpendicular to the axial direction; the plurality of concentric ridges being distributed over an axial length that is greater when the valve seal is in the deformed shape than in the more relaxed shape, the valve seal having an outer radial periphery that is greater in the more relaxed shape than in the deformed shape.

9. The bifluidic valve of claim 8, wherein the surface of the valve seal comprises a plurality of incremental steps that create the plurality of concentric ridges.

10. The bifluidic valve of claim 8, wherein the surface of the valve seal is wavy to create the plurality of concentric ridges.

11. The bifluidic valve of claim 10, wherein the valve housing at least partially defines a liquid inlet and a liquid outlet, the valve spool is movable in the axial direction relative to the valve housing between home position and depressed position, the valve spool in the depressed position establishes fluid communication between the liquid inlet and the liquid outlet to configure the bifluidic valve in the irrigating configuration, the valve spool in the home position obstructs fluid communication between the liquid inlet and the liquid outlet, and the valve seal moves in the axial direction with the valve spool as the valve spool moves between the home position and the depressed position.

12. The bifluidic valve of claim 11, wherein the valve spool at least partially defines a vent that can be selectively covered and uncovered manually, the vent when uncovered connects the gas inlet in fluid communication with atmosphere, the vent when covered blocks fluid communication between the gas inlet and the atmosphere, and the bifluidic valve is configured in a vented configuration when the vent is uncovered while the valve spool is in the home position.

13. A bifluidic valve for use in an endoscope, the bifluidic valve being configurable selectively to an insufflating configuration and an irrigating configuration, the bifluidic valve comprising:
a valve housing with an inner surface at least partially defining a housing interior, the valve housing at least partially defining a gas inlet and a gas outlet 4;
a valve spool being elongate to define an axial direction, the valve spool extending into the housing interior, the valve spool defining a gas passageway between the gas inlet and the gas outlet 4;
a valve seal encircling the valve spool and being attached thereto, the valve seal being resiliently expandable from a more relaxed shape to a deformed shape in response to a pressure differential between the gas inlet and the gas outlet exceeding a predetermined threshold;
when the bifluidic valve is in the insufflating configuration, the valve seal is in the deformed shape to open the gas passageway and thereby connect the gas inlet in fluid communication with the gas outlet; and
when the bifluidic valve is in the irrigating configuration, the valve seal is in the more relaxed shape to obstruct the gas passageway and thereby block fluid communication between the gas inlet and the gas outlet.

14. The bifluidic valve of claim 13, wherein the valve seal includes a fixed end that is substantially stationary relative to the valve spool, the valve seal includes a movable end that is closer to the valve spool when the valve seal is in the more relaxed shape than when the valve seal is in the deformed shape.

15. The bifluidic valve of claim 13, wherein the valve seal further comprises a flange extending radially from the valve spool, the valve spool is movable in the axial direction between a home position and a depressed position relative to the valve housing, and the flange is in sliding contact with the inner surface of the valve housing when the valve spool moves between the home position and the depressed position.

16. The bifluidic valve of claim 13, wherein the valve seal includes a fixed end that is substantially stationary relative to the valve spool, a movable end that is closer to the valve spool when the valve seal is in the more relaxed shape than when the valve seal is in the deformed shape, and a flange extending radially from the valve spool and contacting the inner surface of the valve housing, and further wherein the valve seal with the fixed end, the movable end and the flange is a seamless unitary piece.

17. The bifluidic valve of claim 16, wherein the flange has an axial thickness that is greater than a radial wall thickness of the valve seal at the movable end.

18. The bifluidic valve of claim 13, wherein the valve seal includes a fixed end, a movable end, and a bulging section axially interposed therebetween, the fixed end is substantially stationary relative to the valve spool, the movable end is closer to the valve spool when the valve seal is in the more relaxed shape than when the valve seal is in the deformed shape, and the bulging section engages the inner surface of the valve housing.

19. The bifluidic valve of claim 18, wherein the valve seal with the fixed end, the movable end and the bulging section is a seamless unitary piece.

20. The bifluidic valve of claim 13, wherein the valve housing at least partially defines a liquid inlet and a liquid outlet, the valve spool is movable in the axial direction between a home position and a depressed position relative to the valve housing, the valve spool in the depressed position establishes fluid communication between the liquid inlet and the liquid outlet, and the valve spool in the home position obstructs fluid communication between the liquid inlet and the liquid outlet.
